(19) United States
(12) Patent Application Publication
(10) Pub. No.: US 2024/0252914 A1
(43) Pub. Date: Aug. 1, 2024

(54) ELECTRONIC DEVICE FOR PROVIDING OR AFFECTING SMELL AND ASSOCIATED EXTENDED REALITY DEVICE

(71) Applicant: City University of Hong Kong, Kowloon (HK)

(72) Inventors: Xinge Yu, Kowloon (HK); Yiming Liu, Kowloon (HK); Wooyoung Park, Kowloon (HK); Chunki Yiu, Kowloon (HK)

(21) Appl. No.: 18/162,049

(22) Filed: Jan. 31, 2023

(51) Int. Cl. A63F 13/28; G06F 3/01

(52) U.S. Cl. CPC A63F 13/28; G06F 3/011

(57) ABSTRACT

An electronic device for providing or affecting a smell. The electronic device includes a device body, which has a chamber for containing one or more chemical substances and an interface through which the one or more chemical substances in the chamber can be released from the device body to provide or affect a smell perceivable by a user. The electronic device also includes a release control mechanism arranged at least partly in the device body and operable to control release of the one or more chemical substances from the device body through the interface.

### TECHNICAL FIELD

The invention relates to an electronic device for providing or affecting smell. The invention also relates to an extended reality device including one or multiple ones of such electronic device.

### BACKGROUND

Electronic device for providing or affecting smell are known. One example of these electronic devices is olfactory displays. Generally, olfactory displays can generate and provide vaporized odor molecules (e.g., from odorant in stock form) to an environment in which a subject is present or to a subject. The vaporized odor molecules can then be processed by an olfactory organ of the subject to identify an associated smell.

### SUMMARY OF THE INVENTION

In a first aspect, there is provided an electronic device for providing or affecting a smell. The electronic device comprises a device body that has a chamber for containing one or more chemical substances and an interface through which the one or more chemical substances in the chamber can be released from the device body to provide or affect a smell perceivable by a user (e.g., animal, human, etc.). The electronic device further comprises a release control mechanism arranged at least partly in the device body and operable to control release of the one or more chemical substances from the device body through the interface.

Optionally, the device body is flexible.

Optionally, the device body comprises a housing that provides the chamber and the interface. The chamber and the interface may be provided by the same housing.

Optionally, the device body comprises a flexible housing that provides the chamber and the interface.

Optionally, the flexible housing comprises, or consists only of, a first end portion, a second end portion opposite the first end portion, and a third portion extending between the first end portion and the second end portion to define at least part of the chamber. The first end portion may be a base portion.

Optionally, the first end portion is flexible. Optionally, the first end portion comprises, or consists only, of a film, which may be flexible.

Optionally, the first end portion is configured for attachment to the user, e.g., to a skin of the user.

Optionally, the second end portion is flexible. Optionally, the first end portion comprises, or consists only of, a film, which may be flexible.

Optionally, the interface is arranged in the first end portion.

Optionally, the flexible housing is made of plastic, e.g., one or more plastic materials.

Optionally, the first end portion is made of plastic, e.g., one or more plastic materials. In some examples, the first end portion is made of polyimide (PI). Optionally, the third portion is made of plastic, e.g., one or more plastic materials. In some examples, the third portion is made of epoxy. Optionally, the second end portion is made of plastic, e.g., one or more plastic materials. In some examples, the second end portion is made of polyethylene terephthalate (PET). Optionally, the third portion is arranged to be more rigid than the first end portion and the second end portion, e.g., by virtue of material choice, construction, structure, etc. Optionally, the second end portion is arranged to be more rigid than the first end portion, e.g., by virtue of material choice, construction, structure, etc.

Optionally, the flexible housing is made partly or entirely of biocompatible material(s).

Optionally, the interface comprises one or more holes through which the one or more chemical substances can pass.

Optionally, the interface comprises a permeable part (e.g., surface, window, etc.) through which the one or more chemical substances can permeate or penetrate.

Optionally, the release control mechanism can be arranged in a first configuration that prevents or reduces release of the one or more chemical substances from the device body through the interface and a second configuration that enables, facilitates, or increases release of the one or more chemical substances from the device body through the interface.

Optionally, the release control mechanism in the first configuration prevents fluid communication between the chamber and the interface to prevent release of the one or more chemical substances from the device body through the interface. Optionally, the release control mechanism in the second configuration enables fluid communication between the chamber and the interface to enable release of the one or more chemical substances from the device body through the interface.

Optionally, the release control mechanism comprises a valve arrangement. Optionally, the valve arrangement comprises: a valve member movable relative to the interface between a blocking state, in which the valve member substantially completely blocks the interface, and a non-blocking state, in which the valve member does not block the interface. The blocking state may generally correspond to the first configuration and the non-blocking state generally corresponds to the second configuration.

In some examples, the valve member is a user-actuable valve member that can be manipulated by a user to change from the blocking state to the non-blocking state and/or from the non-blocking state to the blocking state.

In some examples, the valve member can be additionally or alternatively controlled by a mechanism to change from the blocking state to the non-blocking state and/or from the non-blocking state to the blocking state.

Optionally, the valve arrangement further comprises: a valve member control mechanism operable to control movement of the valve member relative to the interface from the blocking state to the non-blocking state and/or from the non-blocking state to the blocking state.

Optionally, the valve member is biased in the blocking state. Optionally, the valve member is biased in the non-blocking state.

Optionally, the valve member is arranged in the device body. Optionally, the valve member is disposed between the chamber and the interface.

Optionally, the valve member comprises a film or membrane. The film or membrane may be made of plastic e.g., one or more plastic materials. In some examples, the film or membrane is made of polyethylene terephthalate (PET). The film or membrane may be elastic, flexible, and/or reversibly-deformable.

Optionally, the valve member control mechanism comprises: a first magnetic arrangement coupled with (e.g., attached to, embedded in, etc.) the valve member; and a second magnetic arrangement operable or controlled to interact with the first magnetic arrangement to affect movement of the valve member relative to the interface.

In some examples, the second magnetic arrangement is arranged or controlled to interact with the first magnetic arrangement in such a way to move the valve member from the blocking state to the non-blocking state and/or from the non-blocking state to the blocking state.

In some examples, the second magnetic arrangement is arranged or controlled to interact with the first magnetic arrangement in such a way to repeatedly move the valve member between the blocking state and the non-blocking state, for generating an air flow at or near the interface to facilitate release of the one or more chemical substances from the device body through the interface.

Optionally, the first magnetic arrangement is arranged in the device body. Optionally, the first magnetic arrangement is disposed between the valve member and the second magnetic arrangement.

Optionally, the first magnetic arrangement comprises a magnet attached to the valve member. The magnet may be a permanent magnet.

Optionally, the second magnetic arrangement is arranged at least partly in the device body or at least partly in the chamber.

Optionally, the second magnetic arrangement comprises an electromagnet. The electromagnet may include a coil electrically connectable or couplable with a power source. The electrically connection or coupling may be a wired connection or coupling. Optionally, the coil comprises a generally flat spiral coil. Optionally, the coil is made of copper (copper wires).

Optionally, the second magnetic arrangement further comprises the power source electrically connected or coupled with the electromagnet to provide power to operate the electromagnet. The power source may be an AC power source.

Optionally, the second magnetic arrangement further comprises a power control arrangement operable to control one or more properties of the power (e.g., frequency, duty cycle, amplitude) provided by the power source to the electromagnet. By controlling the one or more properties of the power, the movement of the valve member (e.g., movement amplitude, frequency, etc.) can be controlled to affect the release of the one or more chemical substances from the device body through the interface. The power control arrangement may include CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries.

Additionally or alternatively, the release control mechanism comprises a temperature adjustment mechanism operable to adjust a temperature in the chamber to affect a rate of release of the one or more chemical substances from the device body through the interface.

Optionally, the temperature adjustment mechanism is at least partly arranged in the device body.

Optionally, the temperature adjustment mechanism comprises a heating mechanism arranged to provide heat to the chamber.

Optionally, the heating mechanism comprises one or more resistive heating elements. Optionally, the one or more resistive heating elements are arranged in the device body or in the chamber. Optionally, the one or more resistive heating elements comprises one or more electrodes. Optionally, the one or more electrodes comprise one or more serpentine electrodes, which may or may not be generally planar. Optionally, the one or more electrodes comprise one or more Au/Cr electrodes. Optionally, the one or more resistive heating elements are disposed on the first end portion of the device body.

Optionally, the temperature adjustment mechanism comprises a cooling mechanism arranged to remove heat from the chamber. The cooling mechanism may include an active cooling mechanism (e.g., electronic heat removal device, coolant system, etc.).

Optionally, the temperature adjustment mechanism comprises both the heating and cooling mechanisms, to provide heat to the chamber and remove heat from the chamber as needed.

Optionally, the release control mechanism further comprises a temperature sensor operable to sense a temperature in the chamber.

Optionally, the temperature sensor comprises a thermistor, which may be a negative-temperature coefficient (NTC) thermistor. Optionally, the temperature sensor is arranged in the device body or in the chamber.

Optionally, the release control mechanism further comprises a temperature control arrangement operably connected with the temperature adjustment mechanism and the temperature sensor to control operation of the temperature adjustment mechanism based on the temperature sensed by the temperature sensor.

Optionally, the temperature control arrangement is arranged in the device body.

The temperature control arrangement may include CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries.

In some examples, the power control arrangement and the temperature control arrangement are provided by the same CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries. In some examples, the power control arrangement and the temperature control arrangement are provided by different CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries.

Optionally, the temperature control arrangement is configured or programmed to: compare the temperature sensed by the temperature sensor with reference temperature data (e.g., one or more temperature values or one or more temperature ranges, optionally for a period of time), and control operation of the temperature adjustment mechanism based on the comparison. In some examples, the temperature control arrangement is configured or programmed to: change an operation of the temperature adjustment mechanism to enable reduction of temperature in the chamber if the temperature in the chamber as sensed by the temperature sensor reaches or exceeds a set temperature value, e.g., for a period of time.

Optionally, the electronic device further comprises a storage element disposed in the chamber for storing or holding the one or more chemical substances. Optionally, the storage element is disposed between the valve arrangement and the temperature adjustment mechanism. Optionally, the storage element comprises cotton, e.g., a layer or lump of cotton.

Optionally, the electronic device further comprises the one or more chemical substances in the chamber. The one or more substances itself/themselves may be odorless or the one or more substances itself/themselves may provide a smell. The smell or odor may be pleasant (e.g., fragrant, scent, aroma, etc.) or unpleasant (e.g., foul, etc.).

Optionally, the one or more chemical substances may be gaseous material(s) that can be released through the interface.

Optionally, the one or more chemical substances may be phase change material(s) that can be vaporized for release through the interface (e.g., the one or more chemical substances can be in liquid phase and/or gas phase, depending on the environment). In some examples, the one or more chemical substances in the chamber can be or have been vaporized by one or more of natural evaporation, airflow, and heat. In some examples, the vapour of the one or more chemical substances in the chamber can be or have been condensed naturally or by cooling. The vapor of the one or more chemical substances can be released from the device body by diffusion, by airflow, etc.

In some examples, the one or more chemical substances can provide or generate an odor to affect the smell perceivable by the user. In some examples, the one or more chemical substances can react or interact with one or more substances in an environment the electronic device is in to affect the smell perceivable by the user. For example, the one or more chemical substances may affect (e.g., reduce, remove, enhance, etc.) an odor present in the environment (in which the electronic device is operating) to affect the smell perceivable by the user.

Optionally, the electronic device is a portable device configured to be carried by the user.

Optionally, the electronic device is a wearable electronic device configured to be worn on or by the user. In some examples, the wearable electronic device is configured to be attached to a skin of the user. For example, the wearable electronic device may include an attachment mechanism for securing the electronic device to the user, an adhesive arrangement for adhering the electronic device to the skin of the user, etc.

Optionally, the electronic device is fanless, i.e., the electronic device does not include any fan for moving air in or around the electronic device.

Optionally, the electronic device is tubeless, i.e., the electronic device does not include any tube.

Optionally, the electronic device is a cordless electronic device.

Optionally, the electronic device is a standalone electronic device.

In some examples, the electronic device is operable as an olfactory display, e.g. an olfactory display for an extended reality device.

Optionally, the electronic device may have one or more additional chambers, separated from the chamber, each for containing one or more chemical substances. Optionally, the electronic device may have one or more additional interfaces, separated from the interface, through which one or more chemical substances in the chamber(s) can be released from the device body.

In a second aspect, there is provided an extended reality device comprising at least one of the electronic device of the first aspect. The extended reality device may be an augmented reality device, a virtual reality device, a mixed reality device, etc. The extended reality device may be in the form of glasses, computer, laptop, headset, phone, tablet computer, desktop computer, etc. The extended reality device may be wearable, i.e., configured to be worn on or by the user. Optionally, the extended reality device includes multiple ones of the electronic device of the first aspect. The multiple ones of the electronic devices may be connected or arranged in an array. The device bodies of the electronic devices may be integral or connected.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

Terms of degree such that “generally”, “about”, “substantially”, or the like, are used, depending on context, to take into account manufacture tolerance, degradation, trend, tendency, imperfect practical condition(s), etc. In some examples, when a value is modified by terms of degree, such as “about”, such expression may include the stated value, e.g., ±20%, ±10%, or ±5%.

As used herein, unless otherwise specified, the terms “connected”, “coupled”, “mounted”, and the like, are intended to encompass both direct and indirect connection, coupling, mounting, etc. Also, unless otherwise specified, the terms “smell”, “odor”, and their equivalents, are intended to cover smell/odor that is/are both pleasant and unpleasant (which can be a subjective).

### BRIEF DESCRIPTION OF THE DRAWINGS

Embodiments of the invention will now be described, by way of example, with reference to the accompanying drawings, in which:

FIG. 1 is a functional block diagram of an electronic device for providing or affecting a smell in embodiments of the invention;

FIG. 2 is a block diagram of an electronic device for providing or affecting a smell in some embodiments of the invention;

FIG. 3A is a schematic diagram (exploded view) of an electronic device for providing or affecting a smell in one embodiment of the invention;

FIG. 3B is a schematic diagram (perspective view) of the electronic device of FIG. 3A;

FIG. 3C is a schematic diagram (perspective view) of the electronic device of FIG. 3A with some components of the electronic device removed;

FIG. 3D is a photograph (perspective view) of an electronic device for providing or affecting a smell fabricated based on the electronic device of FIG. 3A;

FIG. 3E is a photograph (perspective view) of the electronic device of FIG. 3D with some components of the electronic device removed;

FIG. 3F is a photograph (side view) of the electronic device of FIG. 3D;

FIG. 4 is a graph showing the change in temperature in the chamber of the electronic device of FIG. 3D as the target temperature increased from room temperature to 55° C. (in 5° C. interval) in one example;

FIG. 5 is a graph showing the relationship between the measured temperature (in the chamber of the electronic device of FIG. 3D) and the target temperature, and the relationship between the target temperature and time, in one example;

FIG. 6 is a graph showing the concentration of ethanol provided at the interface of the electronic device of FIG. 3D when the temperature in the chamber of the electronic device of FIG. 3D increased from 35° C. to 55° C. in 5° C. interval (the chamber contains 0.4 mL 95% ethanol solution), in one example;

FIG. 7 is a graph showing the concentration of ethanol provided at the interface of the electronic device of FIG. 3D for different AC power frequencies provided to the electromagnetic coil of the electronic device of FIG. 3D in one example;

FIG. 8 is a graph showing the temperature in the chamber of the electronic device of FIG. 3D for different AC power frequencies provided to the electromagnetic coil of the electronic device of FIG. 3D in one example;

FIG. 9 is a graph showing the concentration of ethanol provided at the interface of the electronic device of FIG. 3D for different AC power amplitudes provided to the electromagnetic coil of the electronic device of FIG. 3D in one example;

FIG. 10 is a graph showing the concentration of ethanol provided at the interface of the electronic device of FIG. 3D for different AC power duty cycles provided to the electromagnetic coil of the electronic device of FIG. 3D in one example;

FIG. 11 is a graph showing the concentration of ethanol provided at the interface of the electronic device of FIG. 3D for different rates of airflow blowing generally perpendicularly to the top surface of the electronic device of FIG. 3D in one example;

FIG. 12 is a graph showing the concentration of ethanol provided at the interface of the electronic device of FIG. 3D against time when airflow initially blows generally perpendicularly to the top surface of the electronic device at a constant airflow rate of about 2.76 m/s and the interface of the electronic device of FIG. 3D becomes blocked at about 0.7s in one example;

FIG. 13 is a graph showing the temperature in the chamber of the electronic device of FIG. 3D during a 12-hour continuous operation of the electronic device (with the target temperature is set at 55° C.) in one example;

FIG. 14 is a plot showing the concentration of ethanol provided by the electronic device of FIG. 3D (operated for over 10 minutes) in 3D space in one example;

FIG. 15 is a plot showing the concentration of ethanol provided by the electronic device of FIG. 3D (operated for over 10 minutes) in 3D space in one example; and

FIG. 16 is a functional block diagram of an extended reality device in some embodiments of the invention.

### DETAILED DESCRIPTION

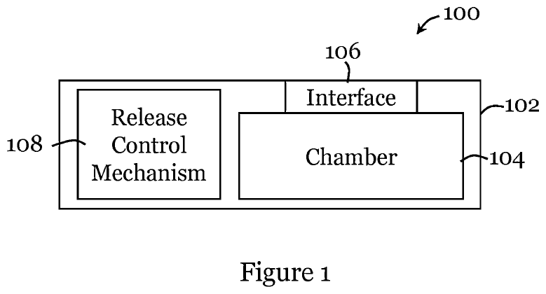

FIG. 1 shows a functional block diagram of an electronic device 100 for providing or affecting a smell in embodiments of the invention. It should be appreciated that FIG. 1 only shows the basic functional components or modules of the electronic device 100 and the electronic device 100 can include additional functional components or modules not specifically illustrated and/or described.

As shown in FIG. 1, the electronic device 100 includes a device body 102 with a chamber 104 that receives, or can receive, chemical substance(s), and an interface 106 through which the chemical substance(s) in the chamber can be released from the device body 102 to provide or affect a smell perceivable by a user. The chamber 104 and the interface 106 can be in fluid communication to allow flow of the chemical substance(s) between the chamber 104 and the interface 106. The chamber 104 and the interface 106 may be arranged close to, e.g., immediately adjacent, each other. The device body 102 may be flexible. In some examples, the device body 102 may include a flexible housing that provides the chamber 104 and the interface 106. The flexible housing may have different housing portions made of the same or different materials. In some examples, the housing are made of plastic (one or more plastic materials). In some examples, the housing are made of biocompatible material(s) which may or may not be plastic. The interface 106 may include one or more holes, or one or more openings, through which the chemical substance(s) can pass, or it may include a permeable surface, window, or like part, through which the chemical substance(s) can permeate or penetrate.

In some examples, the chemical substance(s) received or receivable in the chamber 104 may be gaseous material(s) that can be released from the device body 102 through the interface 106. In some examples, the chemical substance(s) received or receivable in the chamber 104 may be phase change material(s) (e.g., with at least liquid and gas phases) that can be vaporized, naturally or assisted, for release from the device body 102 through the interface 106 by natural diffusion, airflow, etc. The electronic device may further include a storage element in the chamber 104 for storing or holding the chemical substance(s).

The chemical substance(s) may have a smell or may be odorless. In some examples, the chemical substance(s) may provide or generate an odor to affect the smell perceivable by the user. In some examples, the chemical substance(s) may react or interact with one or more substances in an environment the electronic device 100 is in to affect the smell perceivable by the user, such as to affect (e.g., reduce, remove, enhance, etc.) an odor present in the environment the electronic device 100 is in to affect the smell perceivable by the user.

As shown in FIG. 1, the electronic device 100 also includes a release control mechanism 108 that can control release of the chemical substance(s) from the device body 102 through the interface 106. The release control mechanism 108 can control the release of the chemical substance(s), e.g., by selectively enabling and disabling the release of the chemical substance(s), by controlling a rate of the release, etc. The release control mechanism 108 is at least partly arranged in the device body 102. In some examples, the release control mechanism 108 can be arranged in a first configuration that prevents or reduces release of the chemical substance from the device body 102 through the interface 106 and a second configuration that enables, facilitates, or increases release of the chemical substance from the device body 102 through the interface 106. In some examples, the release control mechanism 108 in the first configuration may prevent fluid communication between the chamber 104 and the interface 106 to prevent release of the chemical substance from the device body 102 through the interface 106, and the release control mechanism 108 in the second configuration enables fluid communication between the chamber 104 and the interface 106 to enable release of the chemical substance from the device body 102 through the interface 106. In some examples, the release control mechanism 108 includes one or more of: a valve mechanism that includes valve(s) and associated control, a temperature adjustment and/or control mechanism for adjusting and/or controlling (heating and/or cooling) temperature in or near the chamber 104, etc.

In some examples, the electronic device 100 is a portable device configured to be carried by the user. In some examples, the electronic device 100 is wearable electronic device configured to be worn on or by the user (e.g., attached to a skin of the user). The electronic device 100 may have no fans and/or tubes. In some examples, the electronic device 100 is a cordless electronic device. In some examples, the electronic device 100 is a standalone electronic device.

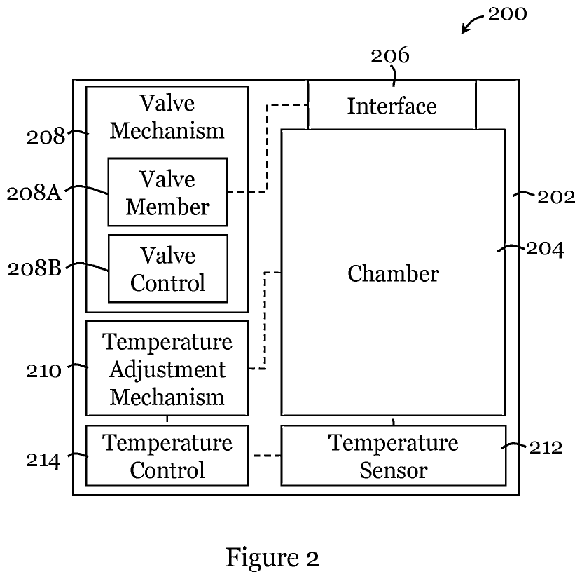

FIG. 2 shows a block diagram of an electronic device 200 for providing or affecting a smell in some embodiments of the invention. It should be appreciated that the electronic device 200 can include additional components or modules not specifically illustrated and/or described. The electronic device 200 can be considered as specific example implementation of the electronic device 100.

As shown in FIG. 2, the electronic device 200 includes a device body 202 with a chamber 204 that receives, or can receive, chemical substance(s), and an interface 206 through which the chemical substance(s) in the chamber can be released from the device body 202 to provide or affect a smell perceivable by a user. The chamber 204 and the interface 206 can be in fluid communication to allow flow of the chemical substance(s) between the chamber 204 and the interface 206. The chamber 204 and the interface 206 may be arranged close to, e.g., immediately adjacent, each other. The device body 202 may be flexible. In some examples, the device body 202 may include a housing that provides the chamber 204 and the interface 206. The housing may be flexible and may include a base portion (end portion), a top portion (another end portion) opposite the base portion, and an intermediate portion extending between the base portion and the top portion. The base portion may be flexible. For example, the base portion may include a flexible film. The base portion may be configured for attachment to the user, e.g., to a skin of the user. The intermediate portion may define at least part of the chamber 204. The top portion may be flexible. For example, the top portion may include a flexible film. The flexible housing may be made partly or entirely of plastic (one or more plastic materials) or of biocompatible material(s), which may or may not be plastic. In some examples, the base portion is made of plastic (e.g., polyimide (PI)). In some examples, the intermediate portion is made of plastic (e.g., epoxy). In some examples, the top portion is made of plastic (e.g., polyethylene terephthalate (PET)). In some examples, the intermediate portion, which provides at least part of the chamber 204, is arranged to be more rigid than the base portion and the top portion. In some examples, the top portion is arranged to be more rigid than the base portion. The relatively rigidity may be controlled by material choice, construction, structure, etc. Different portions of the housing may be integrally formed, molded, or otherwise bonded or attached to each other using bonding agent (e.g., adhesive). The interface 206 may include one or more holes, or one or more openings, through which the chemical substance(s) can pass, or it may include a permeable surface, window, or like part, through which the chemical substance(s) can permeate or penetrate. In some examples, the interface 206 is arranged in the top portion of the housing.

In some examples, the chemical substance(s) received or receivable in the chamber 204 may be gaseous material(s) that can be released from the device body 202 through the interface 206. In some examples, the chemical substance(s) received or receivable in the chamber 204 may be phase change material(s) (e.g., with at least liquid and gas phases) that can be vaporized, naturally or assisted, for release from the device body 202 through the interface 206 by natural diffusion, airflow, etc. The electronic device may further include a storage element in the chamber 204 for storing or holding the chemical substance(s). The storage element may be made of cotton.

The chemical substance(s) itself/themselves may have a smell or may be odorless. In some examples, the chemical substance(s) may provide or generate an odor to affect the smell perceivable by the user. In some examples, the chemical substance(s) may react or interact with one or more substances in an environment the electronic device 200 is in to affect the smell perceivable by the user, such as to affect (e.g., reduce, remove, enhance, etc.) an odor present in the environment the electronic device 200 is in to affect the smell perceivable by the user.

The electronic device 200 also includes a release control mechanism that can control release of the chemical substance(s) from the device body 202 through the interface 206. The release control mechanism can control the release of the chemical substance(s), e.g., by selectively enabling and disabling the release of the chemical substance(s), by controlling a rate of the release, etc. The release control mechanism is at least partly arranged in the device body 202.

As shown in FIG. 2, in some embodiments of the electronic device 200, the release control mechanism includes a valve mechanism 208. The valve mechanism 208 includes a valve member 208A and a valve control 208B. The valve member 208A may be disposed upstream, downstream, or at the interface 206. In some examples, the valve member 208A may be disposed in the device body 202, e.g., between the chamber 204 and the interface 206. The valve member 208A may be movable relative to the interface 206 between a blocking state, in which the valve member 208A substantially completely blocks the interface 206, and a non-blocking state, in which the valve member 208A does not block the interface 206. The valve control 208B is operable to control movement of the valve member 208A relative to the interface 206 from the blocking state to the non-blocking state and/or from the non-blocking state to the blocking state. In some examples, the valve member 208A may be controlled and/or moved by the valve control 208B to change from the blocking state to the non-blocking state and/or from the non-blocking state to the blocking state. In some examples, the valve member 208A can be manually manipulated by a user to change from the blocking state to the non-blocking state and/or from the non-blocking state to the blocking state. The valve member may be biased in the blocking state or in non-blocking state, naturally by material/construction of the valve member 208A or by a biasing means (mechanical, electrical, electromagnetic, etc.). In some examples, the valve member 208A may include a film or membrane, which may be made of plastic (such as polyethylene terephthalate (PET)), and which may be elastic, flexible, and/or reversibly-deformable. In some examples, the valve control may include a magnetic control mechanism, which includes a first magnetic arrangement coupled with (e.g., attached to, embedded in, etc.) the valve member 208A and a second magnetic arrangement operable or controlled to interact with the first magnetic arrangement to affect movement of the valve member 208A relative to the interface 206, e.g., to move the valve member 208A from the blocking state to the non-blocking state and/or from the non-blocking state to the blocking state. For example, the second magnetic arrangement may be arranged or controlled to interact with the first magnetic arrangement in such a way to repeatedly move the valve member 208A between the blocking state and the non-blocking state, for generating an air flow at or near the interface 206 to facilitate release of the chemical substance(s) from the device body 202 through the interface 206. The first magnetic arrangement may be arranged in the device body 202 and disposed between the valve member 208A and the second magnetic arrangement. In some examples, the first magnetic arrangement includes a magnet (e.g., permanent magnet). The second magnetic arrangement may be arranged at least partly in the device body 202 or at least partly in the chamber 204. In some examples, the second magnetic arrangement includes an electromagnet with a coil electrically connectable or couplable (e.g., via wires, not illustrated) with a power source, e.g., an AC power source. The AC power source may be arranged at least partly in the device body 202. In some examples, the second magnetic arrangement also has a power control (e.g., implemented using CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries) operable to control one or more properties of the power (e.g., frequency, duty cycle, amplitude) provided by the power source to the electromagnet to control movement of the valve member 208A (e.g., movement amplitude, frequency, etc.).

As shown in FIG. 2, in some embodiments of the electronic device 200, the release control mechanism also includes a temperature adjustment mechanism 210. The temperature adjustment mechanism 210 is operable to adjust a temperature in the chamber 204 to affect a rate of release of the chemical substance(s) from the device body 202 through the interface 206. The temperature adjustment mechanism 210 is at least partly arranged in the device body 202. The temperature adjustment mechanism may include a heating mechanism, a cooling mechanism, or both (separated or integrated). For example, the temperature adjustment mechanism may include a heating mechanism operable to provide heat to the chamber, to facilitate or increase evaporation or vaporization of the chemical substance(s). For example, the temperature adjustment mechanism may include a cooling mechanism operable to remove heat from the chamber 204, to prevent or reduce evaporation or vaporization of the chemical substance(s). The heating mechanism may be implemented using resistive heating element(s), which may be disposed in the device body 202 or in the chamber 204. The resistive heating element(s) may include electrode(s), such as serpentine electrode(s), and which may or may not be generally planar. The cooling mechanism may be implemented using active cooling system (e.g., electronic heat removal device, coolant system, etc.) or passive cooling system (e.g., cooling fins).

As shown in FIG. 2, in some embodiments of the electronic device 200, the release control mechanism also includes a temperature sensor 212 and a temperature control 214 (e.g., implemented using CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries) operably connected with the temperature sensor 212 and the temperature adjustment mechanism 210. The temperature sensor 212 is operable to sense a temperature in the chamber 204. The temperature sensor 212 may be disposed in the device body 202 or in the chamber 204. The temperature sensor 212 may be implemented using a thermistor, such as a negative-temperature coefficient (NTC) thermistor. The temperature control 214 is operable to control operation of the temperature adjustment mechanism 210 based on the temperature sensed by the temperature sensor 212. The temperature control 214 may be arranged in the device body 202. The temperature control 214 may be configured or programmed to: compare the temperature sensed by the temperature sensor 212 with reference temperature data (e.g., one or more temperature values or ranges, optionally for a period of time), and control operation of the temperature adjustment mechanism 210 based on the comparison. For example, the temperature control 214 may change an operation of the temperature adjustment mechanism 210 to facilitate reduction of or reduce the temperature in the chamber 204 if the temperature in the chamber 204 as sensed by the temperature sensor 212 reaches or exceeds a set temperature value, e.g., for a period of time.

In some examples, at least some of the valve control 208B (power control) and at least some of the temperature control 214 are provided by the same controller(s) and/or circuit(s) in the electronic device 200. In some examples, the valve control 208B (power control) and the temperature control 214 are provided by different controller(s) and/or circuit(s) in the electronic device 200. The operations of the valve control 208B and the temperature control 214 can be synchronized or coordinated by controller(s) and/or circuit(s) of the electronic device 200.

In some examples, the electronic device 200 may include a communication interface, e.g., a wireless communication interface, for communicating data, information, instruction, etc., with another electronic device. For example, the electronic device 200 may receive operation command from another electric device through the communication interface to affect operation of the valve mechanism 208 and/or the temperature adjustment mechanism 210.

In some examples, the electronic device 200 is a portable device configured to be carried by the user. In some examples, the electronic device 200 is wearable electronic device configured to be worn on or by the user (e.g., attached to a skin of the user). The electronic device 200 may have no fans and/or tubes. In some examples, the electronic device 200 is a cordless electronic device. In some examples, the electronic device 200 is a standalone electronic device.

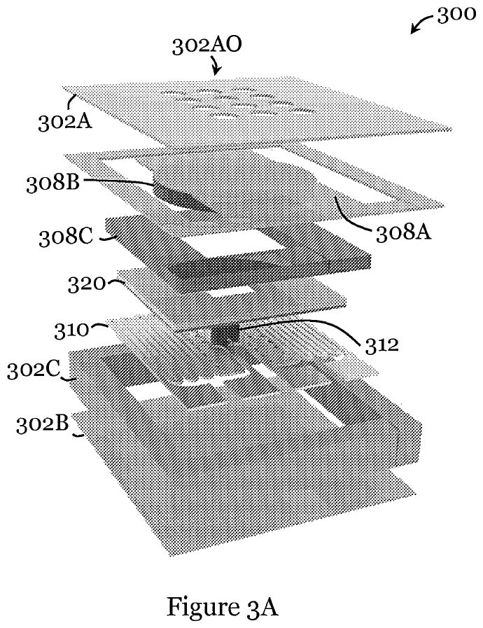

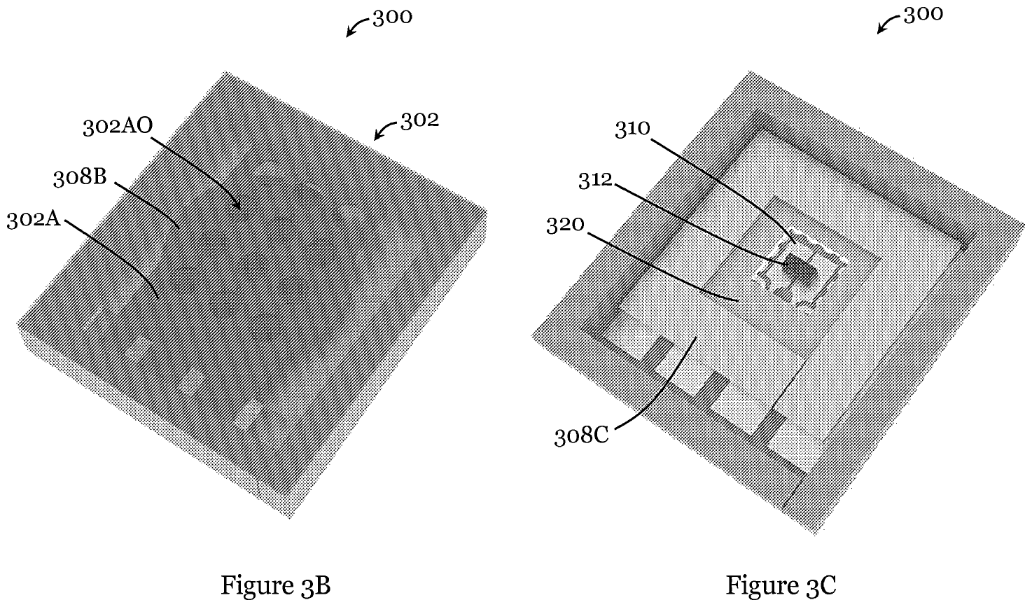

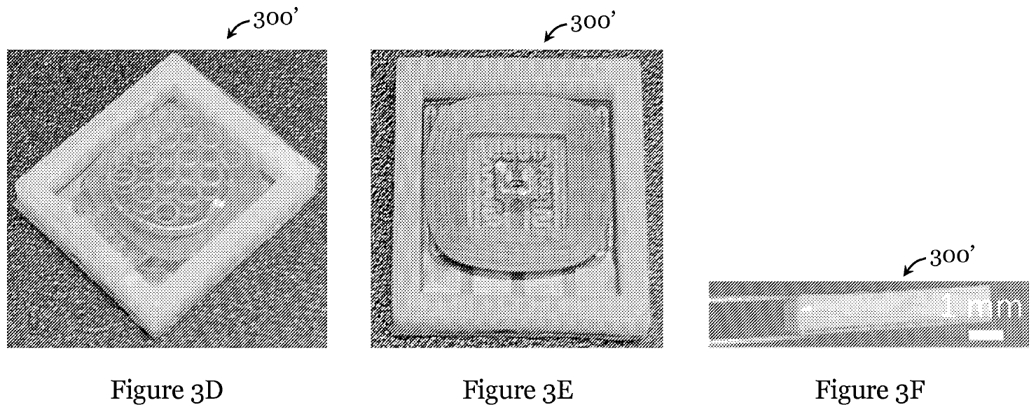

FIGS. 3A to 3C show an electronic device 300 for providing or affecting a smell in one embodiment of the invention. The electronic device 300 can be considered as a specific example implementation of the electronic device 100 or the electronic device 200.

As shown in FIGS. 3A to 3C, the electronic device 300 includes a device body 302 that includes a flexible housing. The flexible housing defines a chamber for containing chemical substance(s) and an interface through which the chemical substance(s) in the chamber can be released from the device body 302 to provide or affect a smell perceivable by a user. In this embodiment, the flexible housing includes a generally rectangular top part 302A with a polyethylene terephthalate (PET) film, a generally rectangular base part 302B with a polyimide (PI) film, and an intermediate frame 302C with a generally rectangular shaped epoxy ring. The top part 302A, the base part 302B (opposite the top part 302A), and the frame 302C may be bonded to or molded with each other, and they together define the chamber. In this embodiment, the base PI part 302B is constructed to be flexible, and may be configured to be attached to a skin of a user. The intermediate frame 302C is constructed to be less flexible than the base part 302B and the top part 302A to provide structural support for the housing. An array of openings 302AO are formed in the top part 302A to provide the interface.

The electronic device 300 also includes a release control mechanism arranged at least partly in the device body 302 and operable to control release of the chemical substance(s) from the device body 302 through the openings 302AO.

As shown in FIGS. 3A to 3C, the release control mechanism includes a magnetically-controlled valve mechanism that includes a flexible polyethylene terephthalate (PET) film 308A disposed beneath the top part 302A and configured to operate as a valve member, a permanent magnet 308B, shaped as a circular disc, attached or coupled to the flexible polyethylene terephthalate (PET) film 308A, and an electromagnetic coil 308C disposed in the chamber and below the permanent magnet 308B. The flexible polyethylene terephthalate (PET) film 308A is sized and shaped such that it can cover the openings 302AO. The flexible polyethylene terephthalate (PET) film 308A is movable up and down between a blocking state, in which it substantially completely blocks the openings 302AO and substantially seals the chamber, and a non-blocking state, in which it does not block the openings 302AO. The permanent magnet 308B is disposed below the upper surface of the flexible polyethylene terephthalate (PET) film 308A, and is arranged to move with the flexible polyethylene terephthalate (PET) film 308A. The electromagnetic coil 308C includes a generally flat spiral coil, made of copper, configured to operate as an electromagnet to control movement of the permanent magnet 308B hence the flexible polyethylene terephthalate (PET) film 308A relative to the openings 302AO. FIG. 3C shows the electronic device 300 without the top part 302A, film 308A, and magnet 308B. In this embodiment, although not illustrated, the electromagnetic coil 308C is electrically connected or coupled with an AC power source and a power control arrangement (e.g., a microcontroller) that controls one or more properties of the power (e.g., frequency, duty cycle, amplitude) provided by the AC power source. The AC power source and/or the power control arrangement may be arranged in the device body. In operation of the magnetically-controlled valve mechanism, power (e.g., an alternating current) is provided to the electromagnetic coil 308C to operate the electromagnetic coil 308C such that it interacts with the permanent magnet 308B to move the flexible polyethylene terephthalate (PET) film 308A between the blocking state and the non-blocking state. The movement (e.g., frequency, amplitude, etc.) of the flexible polyethylene terephthalate (PET) film 308A can be controlled by the power provided to the electromagnetic coil 308C. The movement of the flexible polyethylene terephthalate (PET) film 308A relative to the openings 302AO may not only selectively prevent and enable release of the chemical substance(s) through the openings 302AO, but may also generate an airflow near the openings 302AO to facilitate release of the chemical substance(s) through the openings 302AO (when the openings 302AO are not blocked).

As shown in FIGS. 3A to 3C, the release control mechanism also includes a temperature adjustment mechanism, a temperature sensor, and a temperature control arrangement. In this embodiment, the temperature adjustment mechanism includes a pair of generally planar serpentine electrodes 310, made of gold/chromium, arranged in or on the PI film of the base part 302B. The generally planar serpentine electrodes 310 can be resistively heated to increase a temperature inside the chamber. In this embodiment, the temperature sensor includes a NTC thermistor 312 disposed on or above the electrodes and in the chamber, for detecting a temperature inside the chamber. The NTC thermistor 312 may extend at least partly through the space defined by the electromagnetic coil 308C. Although not illustrated, the temperature control arrangement (e.g., a microcontroller), electrically connected with the electrodes 310 and the NTC thermistor 312, may be disposed in the device 300 or the chamber. The temperature control arrangement is configured or programmed to compare the temperature sensed by the NTC thermistor 312 with reference temperature data (e.g., one or more temperature values or ranges), and control operation of (e.g., activate, deactivate) the electrodes 310 based on the comparison.

In this embodiment, the properties of the chemical substance(s) used in the electronic device 300 may be the same as those described with reference to the electronic devices 100, 200, hence are not repeated here. In this embodiment, as shown in FIG. 3A, a layer of cotton 320 is disposed between the electrodes 310 and the electromagnetic coil 308C for storing or holding the chemical substance(s) in liquid form prior to vaporization.

It should be appreciated that the release of the chemical substance(s) from the device body 302 through the openings 302AO may be influenced by temperature in the chamber, the operation of the valve mechanism, a surrounding airflow rate, etc.

FIGS. 3D to 3F show an electronic device 300′ for providing or affecting a smell, fabricated in accordance with the design of the electronic device 300. The electronic device 300′ has generally the same structure as the electronic device 300. FIG. 3F illustrates the thickness of the electronic device 300′ (held by forceps) in millimeter scale (about 1 mm).

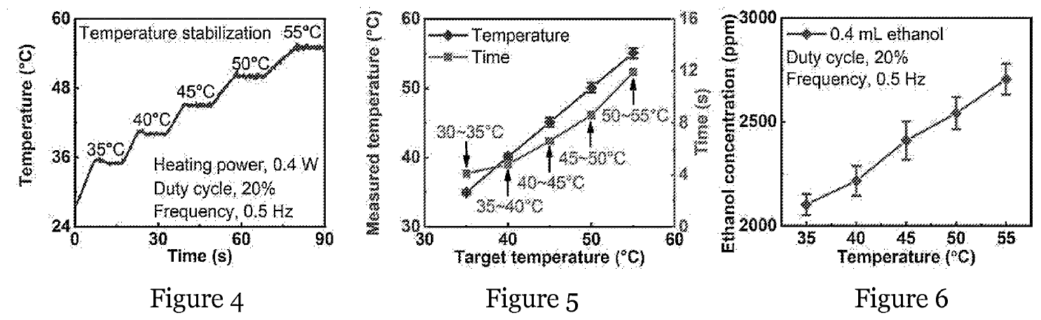

Experiments are performed to test the performance of the electronic device in FIGS. 3D to 3F. Results of these experiments are shown in FIGS. 4 to 15 as examples. In the experiments, unless otherwise specified, ethanol solution is used as the example chemical substance as it is odorous, biocompatible, can be sensed by available commercial sensor with high response rate, and has relatively high evaporation rate.

FIGS. 4 and 5 show the temperature response of the device as the target temperature in the chamber (set in the temperature controller) is increased from room temperature to 55° C. in an interval of 5° C., while the heating power to the electrodes are fixed at 0.4 W and the AC power to the coil (electromagnetic coil) are about 0.5 Hz and has a duty cycle of about 20%.

FIG. 4 shows the temperature stabilization results.

In the test for obtaining the results in FIG. 5, the electrodes are controlled to operate and the temperature controller is programmed such that when the temperature sensor senses the temperature in the chamber is higher than a set temperature, the temperature controller removes power to the electrodes until the temperature sensor senses that the temperature in the chamber is below the set temperature. As shown in FIG. 5, the temperature fluctuation during such operation is negligible, e.g., less than ±1° C. at the target temperature of 55° C.

FIG. 6 shows the concentration of ethanol generated by the device around the openings of the interface as a function of operation temperature in the chamber ranging from 35° C. to 55° C., with 0.4 mL 95% ethanol solution injected into the chamber.

Tests are performed to further investigate the effect of the frequency of the AC power provided to the coil on the concentration of ethanol generated by the device around the openings of the interface. In these tests, the AC power frequencies applied are 0.5 Hz, 1 Hz, and 2 Hz for the electromagnetic coil at a duty cycle of about 20%.

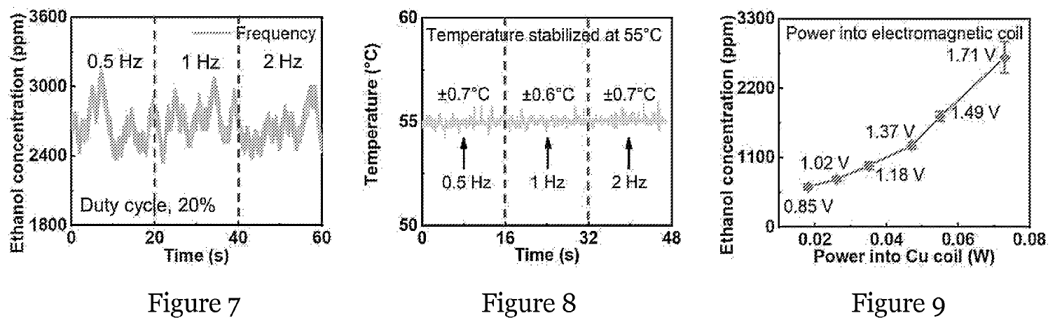

FIG. 7 shows the concentration of ethanol generated by the device around the openings of the interface when different AC power frequencies are applied to the coil whereas FIG. 8 shows the corresponding temperature in the chamber when these different AC power frequencies are applied. From FIGS. 7 and 8, it can be observed that the average ethanol concentration generated by the device around the openings in the three AC power frequencies are generally the same, at around 2700 ppm, and the temperature in the chamber remains relatively constant at 55° C. It can thus be determined that the AC power frequency has little effect on the concentration of ethanol generated by the device around the openings.

Tests are performed to further investigate the effect of the amplitude of the AC power provided to the coil on the concentration of ethanol generated by the device around the openings of the interface. It can be expected that the AC power amplitude may affect the movement amplitude of the permanent magnet hence the valve (PET film). In these tests, the AC power applied are at a duty cycle of about 20%.

FIG. 9 shows the concentration of ethanol generated by the device around the openings of the interface when different AC power amplitudes are applied to the coil. As shown in FIG. 9, an increase in power or power amplitude would increase the concentration of ethanol generated. At the power of 0.077 W, the concentration of ethanol generated is 2717ppm.

Tests are performed to further investigate the effect of the duty cycle of the AC power provided to the coil on the concentration of ethanol generated by the device around the openings of the interface. In these tests, the duty cycles applied to the coil are 20%, 40%, 60%, and 80%.

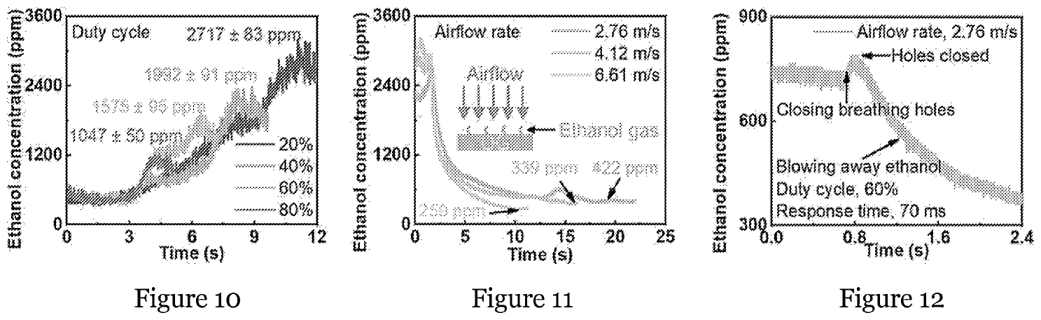

FIG. 10 shows the concentration of ethanol generated by the device around the openings of the interface when different AC power duty cycles are applied to the coil. As shown in FIG. 10, AC power with a 20% duty cycle has a longer ethanol release time than AC power with a 80% duty cycle. Also, as the AC power duty cycle increases from 20% to 80%, the ethanol concentration drops from 2717 ppm to 1047 ppm.

From the above, it can be seen that the amplitude and duty cycle of the AC power to the coil (hence movement of the valve) can be manipulated to provide a relatively fast response time (with less delay, faster than the temperature control mechanism) in terms chemical substance(s) release.

Tests are performed to further investigate the effect of flow rate of air blowing generally perpendicularly to the top surface of the device on the concentration of ethanol generated by the device around the openings of the interface. In these tests, the flow rates of 2.76 m/s, 4.12m/s and 6.61 m/s are used.

FIG. 11 shows the concentration of ethanol generated by the device around the openings of the interface when different flow rates of air are blowing generally perpendicularly to the top surface of the device. As shown in FIG. 11, a higher flow rate can lead to a lower stabilized ethanol concentration.

Tests are performed to further investigate the effect of the blocking of the openings of the interface on the concentration of ethanol generated by the device around the openings of the interface. In these tests, air with a constant airflow rate of 2.76 m/s are continuously blown generally perpendicularly to the top surface of the device and the openings becomes blocked at about 0.7 s. FIG. 12 shows the results. As shown in FIG. 12, after the openings are blocked at about 0.7 s, the ethanol concentration starts to increase for 70 ms and then drops as time passes. The initial increase may be induced by motion of valve (film) as it blocks the openings.

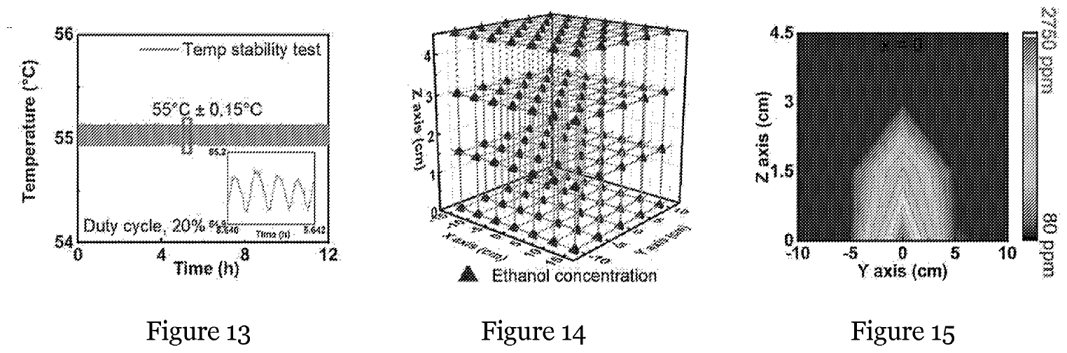

Tests are performed to further investigate the stability of operation of the electronic device. In these tests, the temperature response of the device (temperature in the chamber) is monitored as the device operates continuously for 12 hours with the target temperature set at 55° C. FIG. 13 shows the results, which indicates that the temperature in the chamber remain relatively stable throughout.

Tests are performed to further investigate the ethanol concentration mapping generated by the device around the openings of the interface, after the device has operated for over 10 minutes (e.g., reaches equilibrium state). FIGS. 14 and 15 show the results, and it can be seen that the ethanol can reach 1.5 cm away from the interface. This relatively short transmission distance could avoid unnecessary transmission in some applications.

The above embodiments of the electronic device can be used in various applications. In some examples, the electronic device can be used for providing olfactory information in immersive environments (virtual reality, augmented reality, mixed reality). In these examples, the electronic device can be used as olfactory display for extended reality (e.g., virtual reality) applications. Adding an olfactory display to extended reality applications could enhance immersion sense of users in a virtual environment, and the precise control or providing of smell could facilitate a more realistic experience for users in such environment. For example, the electronic device can be integrated into visual virtual reality systems or devices, and can be operated or driven by software commands to enable immersive environments that can provide synchronized or un-synchronized visual and olfactory virtual reality. In some industries such as defense, fire protection, oil and gas, and aviation, incorporating olfactory perception into virtual reality technology can enhance situational realism and facilitate hazard identification analysis. In some examples, the electronic device can be used to complement a mass audio-visual entertainment experience, such as a film, a television program, a computer games, etc. In some examples, the electronic device can be mounted or used in movie theaters and amusement arcades. In some examples, the electronic device can be used or implemented as wearable (e.g., skin worn or attached) electronics. In some examples, the electronic device can be used for olfactory-driven training and therapy, e.g., for providing specific odor(s) to a user to alter an emotional state of the user (e.g., relieving stress, concentration, relax, etc.). In some examples, the electronic device can be used as a medical device in aromatherapy, which may be combined with virtual reality therapy to treat psychological disorder such as depression and phobia and improve the immune function of users or patients.

The above embodiments of the electronic device may have one or more of the following advantages. The above embodiments of the electronic device may have one or more additional or alternative advantages not described. In some examples, the electronic device can be used as a standalone device, not mounted or installed to another device. In some examples, the electronic device can be flexed, and less prone to damage. In some examples, the electronic device can be made small and/or compact, hence portable or even wearable. In some examples, the electronic device can be arranged to be directly attachable to a skin of a user. In some examples, the electronic device can provide a widespread smell. In some examples, the electronic device can provide a long-lasting smell. In some examples, the electronic device can alter smell (e.g., eliminated odor) present in an environment. In some examples, the electronic device use phase change material(s) as the chemical substance(s), the release and removal of which can be better controlled.

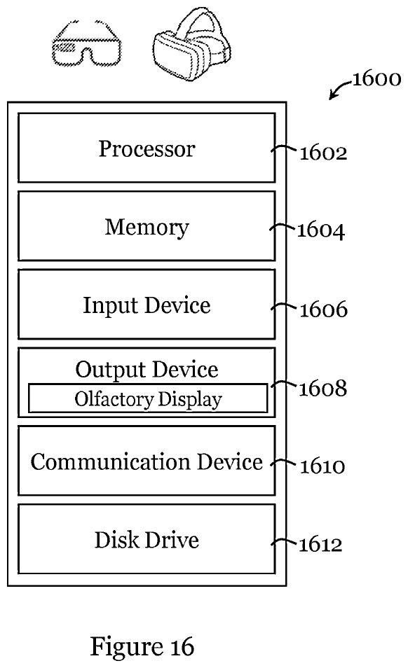

FIG. 16 shows an extended reality device 1600 in some embodiments of the invention. The extended reality device 1600 can be integrated with or otherwise operably coupled with the electronic device of the invention (such as electronic devices 100, 200, 300). The extended reality device 1600 generally comprises suitable components necessary to receive, store, and execute appropriate computer instructions, commands, and/or codes. The main components of the extended reality device 1600 are a processor 1602 and a memory (storage) 1604. The processor 1602 may include one or more: CPU(s), MCU(s), GPU(s), logic circuit(s), Raspberry Pi chip(s), digital signal processor(s) (DSP), application-specific integrated circuit(s) (ASIC), field-programmable gate array(s) (FPGA), and/or any other digital or analog circuitry/circuitries configured to interpret and/or to execute program instructions and/or to process signals and/or information and/or data. The memory 1604 may 1604 may include one or more volatile memory (such as RAM, DRAM, SRAM, etc.), one or more non-volatile memory (such as ROM, PROM, EPROM, EEPROM, FRAM, MRAM, FLASH, SSD, NAND, NVDIMM, etc.), or any of their combinations. Appropriate computer instructions, commands, codes, information and/or data may be stored in the memory 1604. The processor 1602 and memory (storage) 1604 maybe integrated or separated (and operably connected). Optionally, the extended reality device 1600 further includes one or more input devices 1606. Examples of such input device 1606 include: keyboard, mouse, stylus, image scanner, microphone, tactile/touch input device (e.g., touch sensitive screen), image/video input device (e.g., camera), etc. Optionally, the extended reality device 1600 further includes one or more output devices 1608. Examples of such output device 1608 include: display (e.g., monitor, screen, projector, etc.), speaker, headphone, earphone, printer, additive manufacturing machine (e.g., 3D printer), etc. The display may include a LCD display, a LED/OLED display, or other suitable display, which may or may not be touch sensitive. In some embodiments, the output device 1608 of the extended reality device 1600 includes at least one of the electronic device of the invention (such as electronic devices 100, 200, 300) as an olfactory display. The processor 1602 may provide instructions and commands to control operation of the olfactory display. The extended reality device 1600 may further include one or more disk drives 1612 which may include one or more of: solid state drive, hard disk drive, optical drive, flash drive, magnetic tape drive, etc. A suitable operating system may be installed in the extended reality device 1600, e.g., on the disk drive 1612 or in the memory 1604. The memory 1604 and the disk drive 1612 maybe operated by the processor 1602. Optionally, the extended reality device 1600 also includes a communication device 1610 for establishing one or more communication links (not shown) with one or more other computing devices, such as servers, personal computers, terminals, tablets, phones, watches, IoT devices, or other wireless computing devices. The communication device 1610 may include one or more of: a modem, a Network Interface Card (NIC), an integrated network interface, a NFC transceiver, a ZigBee transceiver, a Wi-Fi transceiver, a Bluetooth® transceiver, a radio frequency transceiver, a cellular (2G, 3G, 4G, 5G, above 5G, or the like) transceiver, an optical port, an infrared port, a USB connection, or other wired or wireless communication interfaces. Transceiver may be implemented by one or more devices (integrated transmitter(s) and receiver(s), separate transmitter(s) and receiver(s), etc.). The communication link(s) may be wired or wireless for communicating commands, instructions, information and/or data. In one example, the processor 1602, the memory 1604 (optionally the input device(s) 1606, the output device(s) 1608, the communication device(s) 1610 and the disk drive(s) 1612, if present) are connected with each other, directly or indirectly, through a bus, a Peripheral Component Interconnect (PCI), such as PCI Express, a Universal Serial Bus (USB), an optical bus, or other like bus structure. In one embodiment, at least some of these components may be connected wirelessly, e.g., through a network, such as the Internet or a cloud computing network. A person skilled in the art would appreciate that the extended reality device 1600 shown in FIG. 16 is merely an example and that the extended reality device 1600 can in other embodiments have different configurations (e.g., include additional components, has fewer components, etc.). The extended reality device 1600 maybe used as a virtual reality device, a mixed reality device, an augmented reality device, etc.

It should be appreciated that where any parts of the devices and systems of the invention are either wholly implemented by computing system or partly implemented by computing systems then any appropriate computing system architecture may be utilized. This will include stand-alone computers, network computers, dedicated or non-dedicated hardware devices. Where the terms “computing system” and “computing device” are used, these terms are intended to include (but not limited to) any appropriate arrangement of computer or information processing hardware capable of implementing the function described.

It will be appreciated by person(s) skilled in the art that numerous variations and/or modifications may be made to the invention as shown in the specific embodiments to provide other embodiments of the invention. The described embodiments of the invention should therefore be considered in all respects as illustrative, not restrictive. Example optional features are provided in the summary and the description. Some embodiments of the invention may include one or more of these optional features (some of which are not specifically illustrated in the drawings). Some embodiments of the invention may lack one or more of these optional features (some of which are not specifically illustrated in the drawings). In some embodiments, the construction of the device may be different from those illustrated. For example, the device body of the electronic device can be of a different shape, size, form, etc. than those illustrated. The device body of the electronic device may include more than one chamber for containing chemical substance(s) and/or more than one interface through which chemical substance(s) in the chamber(s) can be released. For example, the release control mechanism may include (i) only the valve mechanism, (ii) only the temperature adjustment mechanism (no sensor and control), (iii) only the temperature adjustment mechanism, temperature sensor and control, etc. The valve mechanism may or may not be magnetically operated. While not clearly illustrated in the Figures associated with some of the above embodiments, the electrical connection or coupling of the components of the electronic device may be via wired means (e.g., wires) or wireless means, as applicable and appropriate. The electronic device may be used as a standalone device, integrated in another device, or attached to another device. The electronic device is not limited in its application to an olfactory display for an extended reality device. In some examples, the response time of the release control mechanism or the valve mechanism may be down to the order of milliseconds.

1. An electronic device for providing or affecting a smell, comprising:
a device body comprising
a chamber for containing one or more chemical substances; and
an interface through which the one or more chemical substances in the chamber can be released from the device body to provide or affect a smell perceivable by a user; and
a release control mechanism arranged at least partly in the device body and operable to control release of the one or more chemical substances from the device body through the interface.

2. The electronic device of claim 1, wherein the device body is flexible.

3. The electronic device of claim 2, wherein the device body comprises a flexible housing that comprises:
a first end portion,
a second end portion opposite the first end portion, and
an third portion extending between the first end portion and the second end portion to define at least part of the chamber.

4. The electronic device of claim 3, wherein the interface is arranged in the second end portion.

5. The electronic device of claim 4, wherein the interface comprises one or more holes through which the one or more chemical substances can pass.

6. The electronic device of claim 1, wherein the release control mechanism can be arranged in:
a first configuration that prevents or reduces release of the one or more chemical substances from the device body through the interface, and
a second configuration that enables, facilitates, or increases release of the one or more chemical substances from the device body through the interface.

7. The electronic device of claim 6,
wherein the release control mechanism in the first configuration prevents fluid communication between the chamber and the interface to prevent release of the one or more chemical substances from the device body through the interface; and
wherein the release control mechanism in the second configuration enables fluid communication between the chamber and the interface to enable release of the one or more chemical substances from the device body through the interface.

8. The electronic device of claim 7, wherein the release control mechanism comprises a valve arrangement that comprises:
a valve member movable relative to the interface between a blocking state, in which the valve member substantially blocks the interface, and a non-blocking state, in which the valve member does not block the interface; and
a valve member control mechanism operable to control movement of the valve member relative to the interface from the blocking state to the non-blocking state and/or from the non-blocking state to the blocking state;
wherein the blocking state generally corresponds to the first configuration and the non-blocking state generally corresponds to the second configuration.

9. The electronic device of claim 8, wherein the valve member comprises a film or membrane arranged in the device body.

10. The electronic device of claim 8, wherein the valve member control mechanism comprises:
a first magnetic arrangement coupled with the valve member; and
a second magnetic arrangement operable or controlled to interact with the first magnetic arrangement to affect movement of the valve member relative to the interface.

11. The electronic device of claim 10, wherein the second magnetic arrangement is arranged or controlled to interact with the first magnetic arrangement in such a way to repeatedly move the valve member between the blocking state and the non-blocking state, for generating an air flow at or near the interface to facilitate release of the one or more chemical substances from the device body through the interface.

12. The electronic device of claim 10, wherein the first magnetic arrangement is arranged in the device body and disposed between the valve member and the second magnetic arrangement.

13. The electronic device of claim 10, wherein the first magnetic arrangement comprises a magnet attached to the valve member.

14. The electronic device of claim 13, wherein the second magnetic arrangement comprises an electromagnet arranged in the device body, and the electromagnet comprises a coil electrically connectable or couplable with a power source.

15. The electronic device of claim 14, wherein the second magnetic arrangement further comprises the power source electrically connected or coupled with the electromagnet to provide power to operate the electromagnet.

16. The electronic device of claim 15, wherein the second magnetic arrangement further comprises a power control arrangement operable to control one or more properties of the power provided by the power source.

17. The electronic device of claim 8, wherein the release control mechanism further comprises a temperature adjustment mechanism operable to adjust a temperature in the chamber to affect a rate of release of the one or more chemical substances from the device body through the interface.

18. The electronic device of claim 1, wherein the release control mechanism comprises a temperature adjustment mechanism operable to adjust a temperature in the chamber to affect a rate of release of the one or more chemical substances from the device body through the interface.

19. The electronic device of claim 18, wherein the temperature adjustment mechanism comprises a heating mechanism arranged to provide heat to the chamber.

20. The electronic device of claim 19, wherein the heating mechanism comprises one or more resistive heating elements arranged in the chamber.

21. The electronic device of claim 20, wherein the one or more resistive heating elements comprises one or more serpentine electrodes.

22. The electronic device of claim 18, wherein the release control mechanism further comprises:
a temperature sensor operable to sense a temperature in the chamber; and
a temperature control arrangement operably connected with the temperature adjustment mechanism and the temperature sensor to control operation of the temperature adjustment mechanism based on the temperature sensed by the temperature sensor.

23. The electronic device of claim 22, wherein the temperature sensor comprises a thermistor arranged in the chamber.

24. The electronic device of claim 22, wherein the temperature control arrangement is configured or programmed to:
compare the temperature sensed by the temperature sensor with reference temperature data, and
control operation of the temperature adjustment mechanism based on the comparison.

25. The electronic device of claim 1, further comprising a storage element disposed in the chamber for storing or holding the one or more chemical substances.

26. The electronic device of claim 1, further comprising the one or more chemical substances in the chamber.

27. The electronic device of claim 26, wherein the one or more chemical substances can provide or generate an odor to affect the smell perceivable by the user.

28. The electronic device of claim 26, wherein the one or more chemical substances can react or interact with one or more substances in an environment the electronic device is in to affect the smell perceivable by the user.

29. The electronic device of claim 1, wherein the electronic device is wearable electronic device configured to be worn on or by the user.

30. An extended reality device comprising at least one of the electronic device of claim 1.
